(19) United States
(12) Patent Application Publication
(10) Pub. No.: US 2026/0174994 A1
(43) Pub. Date: Jun. 25, 2026

(54) STAGGERED STRAIN RELIEF ARTICULATING JOINT

(71) Applicant: Boston Scientific Scimed, Inc., Maple Grove, MN (US)

(72) Inventors: Emily Luecke, Osseo, MN (US); Brent Dolan, Blaine, MN (US); Joseph Geissler, Minneapolis, MN (US); Shane Graham, Coon Rapids, MN (US)

(21) Appl. No.: 19/421,572

(22) Filed: Dec. 16, 2025

Related U.S. Application Data
(60) Provisional application No. 63/738,251, filed on Dec. 23, 2024.

(51) Int. Cl. A61M 25/01; A61M 25/00

(52) U.S. Cl. CPC A61M 25/0138; A61M 25/0136; A61M 25/0147; A61M 2025/0059

(57) ABSTRACT

A medical device includes a handle and a tubular shaft. The tubular shaft has a proximal portion extending from the handle and a distal portion having a distal end and a deflection region. The shaft defines a longitudinal axis and includes an outer tubular jacket and an articulation member disposed within the jacket in the deflection region. The articulation member has a tube that defines a plurality of openings. The openings are arranged in a first array and a second array, the openings in the first array are diametrically opposed to the openings in the second array, the openings in the first array do not circumferentially overlap the openings in the second array, and the first array is longitudinally offset from the second array.

### CROSS REFERENCE TO RELATED APPLICATIONS

This application claims priority to U.S. Provisional Patent Application No. 63/738,251 entitled “STAGGERED STRAIN RELIEF ARTICULATING JOINT,” filed December 23, 2024, which is hereby incorporated by reference in its entirety.

### TECHNICAL FIELD

The present invention relates to medical devices and methods for catheters for medical procedures. More specifically, the invention relates to devices and methods that include directional enhancement for catheters such as steerable catheters.

### BACKGROUND

Various medical procedures involve catheters inserted into a patient's vasculature. In certain procedures, the catheter may be navigated through the vasculature to a target location in the body. The distal end of the catheters may be inserted into the patient's heart chambers in, for example, interventional electrophysiology procedures. The distal end of the catheter may include one or more electrodes that are used to delivery therapy (e.g., ablation) or map the surface of the heart tissue (e.g., identify the locations of heart tissue that are a source of the arrhythmias). Steering such a catheter can involve controlled bending at the distal end, which can cause failures of the catheter over time and/or due to challenging anatomy.

### SUMMARY

In Example 1, a medical device comprising a handle and a tubular shaft having a proximal portion extending from the handle, and a distal portion having a distal end and a deflection region. The shaft defines a longitudinal axis and includes an articulation member disposed within the deflection region, the articulation member having a tube that defines a plurality of openings, wherein the openings are arranged in a first array and a second array, the openings in the first array are diametrically opposed to the openings in the second array, the openings in the first array do not circumferentially overlap the openings in the second array, and the first array is longitudinally offset from the second array.

In Example 2, the medical device of Example 1, wherein the openings have a stress relieving shape.

In Example 3, the medical device of Example 2, wherein the openings are bulb-shaped, inverted-T-shaped, and/or lollipop-shaped.

In Example 4, the medical device of Example 2, wherein each opening comprise a set of sides and a circular or obround cutout, wherein a width of the circular or obround cutout is wider than a distance between the sides where the sides meet the circular or obround cutout.

In Example 5, the medical device of Example 1, wherein each of the openings comprises a set of sides, each of a first set of the openings has a first gap between the set of sides and each of a second set of the openings has a second gap between the set of sides, wherein the first gap is smaller than the second gap.

In Example 6, the medical device of Example 5, wherein the first gap is at least five times smaller than the second gap.

In Example 7, the medical device of Example 5, wherein the first gap is substantially zero.

In Example 8, the medical device of Example 5, wherein first array of openings comprises the first set of the openings and the second set of openings.

In Example 9, the medical device of Example 5, wherein the articulation member is configured to assume a first curved shape when a first deflection force is applied to the tubular shaft, the articulation member is configured to assume a second curved shape when a second, diametrically opposed deflection force is applied to the tubular shaft, and a first radius of curvature of the first curved shape is smaller than a second radius of curvature of the second curved shape.

In Example 10, the medical device of Example 1, wherein the first array of openings comprises at least 20% more openings than the second array of openings.

In Example 11, the medical device of Example 10, wherein the first array of openings comprises at about 100% more openings than the second array of openings.

In Example 12, the medical device of Example 11, wherein the articulation member is configured to assume a first curved shape when a first deflection force is applied to the tubular shaft, the articulation member is configured to assume a second curved shape when a second, diametrically opposed deflection force is applied to the tubular shaft, and a first radius of curvature of the first curved shape is smaller than a second radius of curvature of the second curved shape.

In Example 13, the medical device of Example 1, wherein the first array is offset such that an opening in the first array is longitudinally positioned halfway between two openings in the second array.

In Example 14, the medical device of any of Examples 1-13, wherein the articulation member includes a first reinforcing member and a second reinforcing member that extend through the tube.

In Example 15, the medical device of any of Examples 1-14, further comprising first and second steering wire lumens extending through the tubular shaft to a location distally beyond the articulation member, and first and second steering wires are received, respectively, within the first and second steering wire lumens, the first and second steering wires each connected to a steering actuator disposed within the handle and to the distal portion of the shaft at a location distal of the articulation region, wherein the first steering wire is configured to apply a first deflection force to the distal portion of the shaft to cause the deflection region to articulate in the first direction, and wherein the second steering wire is configured to apply a second deflection force to the distal portion of the shaft to cause the deflection region to articulate in the second direction.

In Example 16, a medical device comprising a handle and a tubular shaft having a proximal portion extending from the handle, and a distal portion having a distal end and a deflection region, the shaft defining a longitudinal axis and including an outer tubular jacket, and an articulation member disposed within the jacket in the deflection region, the articulation member having a tube that defines a plurality of openings. The openings are arranged in a first array and a second array, wherein the openings in the first array are diametrically opposed to the openings in the second array, the openings in the first array do not circumferentially overlap the openings in the second array, and the first array is longitudinally offset from the second array.

In Example 17, the medical device of Example 16, wherein the openings have a stress relieving shape.

In Example 18, the medical device of Example 17, wherein the openings are bulb-shaped, inverted-T-shaped, and/or lollipop-shaped.

In Example 19, the medical device of Example 16, wherein each of the openings comprises a set of sides, each of a first set of the openings has a first gap between the set of sides, and each of a second set of the openings has a second gap between the set of sides, wherein the first gap is smaller than the second gap.

In Example 20, the medical device of Example 19, wherein the first gap is substantially zero.

In Example 21, the medical device of Example 19, wherein first array of openings comprises the first set of the openings and the second set of openings.

In Example 22, the medical device of Example 16, wherein the first array of openings comprises at least 20% more openings than the second array of openings.

In Example 23, the medical device of Example 16, further comprising first and second steering wire lumens extending through the tubular shaft to a location distally beyond the articulation member, and first and second steering wires received, respectively, within the first and second steering wire lumens, the first and second steering wires each connected to a steering actuator disposed within the handle and to the distal portion of the shaft at a location distal of the articulation region, wherein the first steering wire is configured to apply a first deflection force to the distal portion of the shaft to cause the deflection region to articulate in the first direction, and wherein the second steering wire is configured to apply a second deflection force to the distal portion of the shaft to cause the deflection region to articulate in the second direction.

In Example 24, a medical device comprising a handle and a tubular shaft having a proximal portion extending from the handle, and a distal portion having a distal end and a deflection region, the shaft defining a longitudinal axis and including an outer tubular jacket, and an articulation member disposed within the jacket in the deflection region, the articulation member having a tube that defines a plurality of openings. The tube comprises a first straight beam, a second straight beam, a first plurality of cylindrical shell sectors that join the first straight beam and the second straight beam, and a second plurality of cylindrical shell sectors that join the first straight beam and the second straight beam. The first plurality of cylindrical shell sectors is diametrically opposed to and longitudinally offset from the second plurality of cylindrical shell sectors.

In Example 25, the medical device of Example 24, wherein the openings have a stress relieving shape.

In Example 26, the medical device of Example 25, wherein the openings are bulb-shaped, inverted-T-shaped, and/or lollipop-shaped.

In Example 27, the medical device of Example 24, wherein, each of the openings comprises a set of sides, each of a first set of the openings has a first gap between the set of sides, and each of a second set of the openings has a second gap between the set of sides, wherein the first gap is smaller than the second gap.

In Example 28, the medical device of Example 27, wherein the first gap is substantially zero.

In Example 29, the medical device of Example 27, wherein the first set of the openings and the second set of openings are defined by first plurality of cylindrical shell sectors.

In Example 30, the medical device of Example 24, wherein the first plurality of cylindrical shell sectors comprises at least 20% more cylindrical shell sectors than the second plurality of cylindrical shell sectors.

In Example 31, the medical device of Example 24, further comprising, first and second steering wire lumens extending through the tubular shaft to a location distally beyond the articulation member, and first and second steering wires received, respectively, within the first and second steering wire lumens, the first and second steering wires each connected to a steering actuator disposed within the handle and to the distal portion of the shaft at a location distal of the articulation region, wherein the first steering wire is configured to apply a first deflection force to the distal portion of the shaft to cause the deflection region to articulate in the first direction, and wherein the second steering wire is configured to apply a second deflection force to the distal portion of the shaft to cause the deflection region to articulate in the second direction.

In Example 32, a medical device comprising a handle and a tubular shaft having a proximal portion extending from the handle, and a distal portion having a distal end and a deflection region, the shaft defining a longitudinal axis and including an outer tubular jacket, and an articulation member disposed within the jacket in the deflection region, the articulation member having a tube that defines a plurality of openings and defines a longitudinally extending array of living hinges, wherein each living hinge includes one of the plurality of openings each living hinge has a opposite orientation from an adjacent living hinge, the plurality of openings are arranged in a first array and a second array, and the openings in the first array do not circumferentially overlap the openings in the second array.

In Example 33, the medical device of Example 32, wherein the openings are bulb-shaped, inverted-T-shaped, and/or lollipop-shaped.

In Example 34, the medical device of Example 32, wherein each of the openings comprises a set of sides, each of a first set of the openings has a first gap between the set of sides, and each of a second set of the openings has a second gap between the set of sides, wherein the first gap is smaller than the second gap.

In Example 35, the medical device of Example 32, wherein the first array of openings comprises at least 20% more openings than the second array of openings.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a diagram illustrating an exemplary clinical setting for treating a heart of a patient using an electrophysiology system, consistent with various aspects of the present disclosure.

FIGS. 2A-2C are perspective views of a catheter, consistent with various aspects of the present disclosure. In FIG. 2A, the catheter is depicted in a straight configuration. In FIG. 2B, the catheter is depicted in a first deflected configuration. In FIG. 2C, the catheter is depicted in a second deflected configuration.

FIG. 3 is a side view of a deflection region of a shaft of the catheter, consistent with various aspects of the present disclosure.

FIGS. 4A-4C are views of an articulation member from the deflection region of the catheter, consistent with various aspects of the present disclosure. FIG. 4A is a top view of the articulation member. FIG. 4B is a cross-sectional view of the articulation member as indicated by line 4B-4B in FIG. 4A. FIG. 4C is a cross-sectional view of the articulation member as indicated by line 4C-4C in FIG. 4A.

FIG. 5 is a side view of the articulation member, consistent with various aspects of the present disclosure.

FIG. 6A is a side view the articulation member, consistent with various aspects of the present disclosure.

FIG. 6B is a side view the articulation member, consistent with various aspects of the present disclosure.

FIGS. 7A-7C are views of an alternative articulation member, consistent with various aspects of the present disclosure. FIG. 7A is a perspective view of the alternative articulation member. FIG. 7B is a side view of the alternative articulation member. FIG. 7C is a top view of the alternative articulation member.

FIG. 8 is a side view an alternative articulation member, consistent with various aspects of the present disclosure.

FIG. 9A is a side-view of a portion of an alternative catheter having the alternative articulation member of FIG. 8, consistent with various aspects of the present disclosure.

FIG. 9B is a side-view of a portion of an alternative catheter having the alternative articulation member of FIG. 8, consistent with various aspects of the present disclosure.

FIG. 10 is a side view an alternative articulation member, consistent with various aspects of the present disclosure.

FIG. 11A is a side-view of a portion of an alternative catheter having the alternative articulation member of FIG. 10, consistent with various aspects of the present disclosure.

FIG. 11B is a side-view of a portion of an alternative catheter having the alternative articulation member of FIG. 10, consistent with various aspects of the present disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

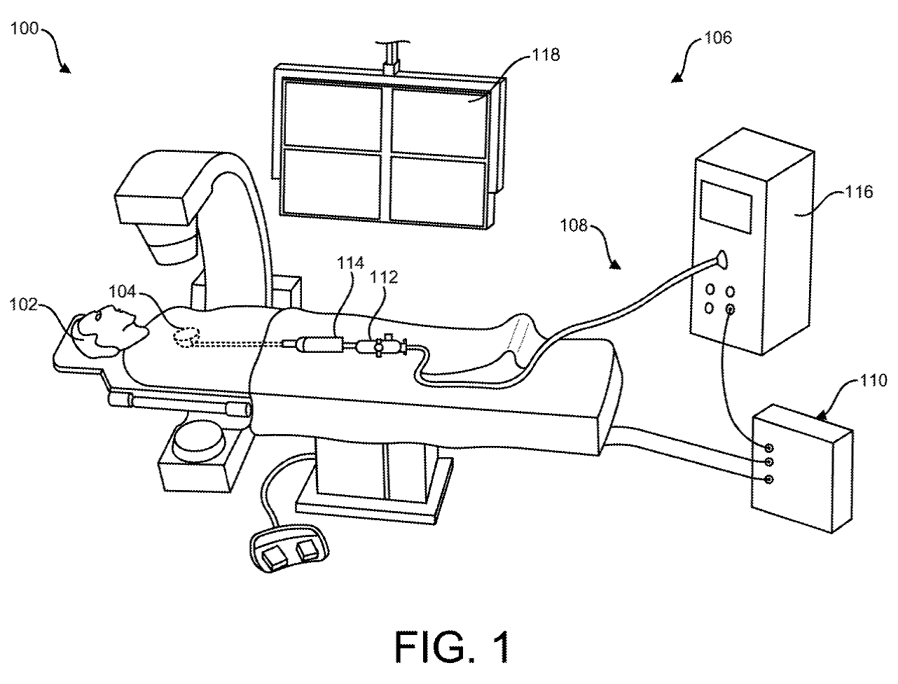

FIG. 1 illustrates an example clinical setting 100 for treating a patient 102, such as for treating a heart 104 of the patient 102, using an electrophysiology system 106. In the illustrated embodiment, the electrophysiology system 106 includes an electroporation catheter system 108 and an electro-anatomical mapping (EAM) system 110. The example electroporation catheter system 108 includes an electroporation catheter 112, an introducer sheath 114, and an electroporation console 116.

In some embodiments, the electroporation console 116 includes a controller, such one or more controllers, processors, or computers, that executes instructions or code, such as processor-executable instructions, out of a non-transitory computer readable medium, such as a memory device, or memory, to cause, such as control or perform, the aspects of the electroporation catheter system 108. Additionally, the electroporation catheter system 108 includes various connecting elements, such as cables, that operably connect the components of the electroporation catheter system 108 to one another and to the components of the EAM system 110.

In the illustrated embodiment, the introducer sheath 114 is operable to provide a delivery conduit through which the electroporation catheter 112 can be deployed to the specific target sites within the patient’s heart 104. Access to the patient’s heart 104 can be obtained through a vessel (not shown), such as a peripheral artery or vein. Once access to the vessel is obtained, the electroporation catheter 112 can be navigated to within the patient’s heart 104, such as within a heart chamber.

In the illustrated embodiment, the electroporation catheter system 108 can be configured to map and/or ablate portions of the patient’s heart 104. When ablating, the electroporation catheter system 108 is configured to deliver ablation electric field energy to targeted tissue in the patient’s heart 104 to create cell death in tissue, for example, rendering the tissue incapable of conducting electrical signals. When mapping, the electroporation catheter system 108 is configured to generate electric fields using the electroporation catheter 112 to create and present, on a display 118, an electro-anatomical map of the patient’s heart 104. In some embodiments, the EAM system 110 includes the OPAL HDx™ mapping system marketed by Boston Scientific Corporation. In some embodiments, the mapping is performed using the INTELLAMAP ORION™ mapping catheter marketed by Boston Scientific Corporation. The mapping aids a physician in planning the ablation prior to delivering ablation electric field energy to the electroporation catheter 112.

The depiction of the electrophysiology system 106 shown in FIG. 1 is intended for illustration or a general overview of the various components of the system 106 and is not intended to imply that the disclosure is limited to any set of components or arrangement of the components. For example, additional hardware components, such as breakout boxes or workstations, can be included in the electrophysiology system 106.

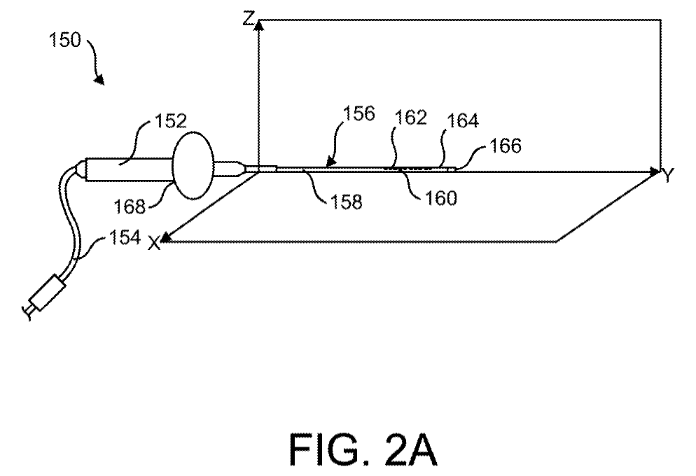

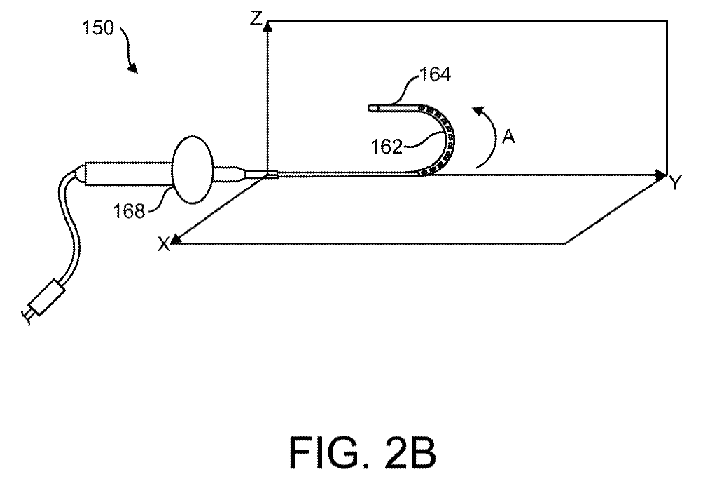

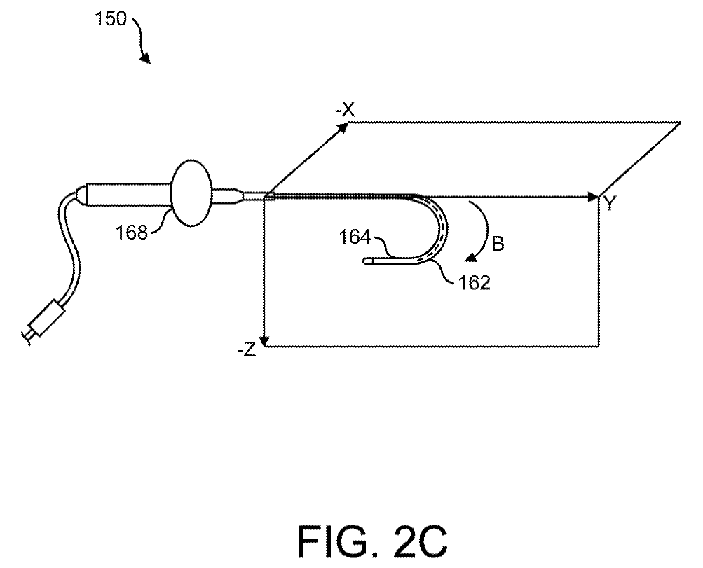

FIG. 2 shows a catheter 150 which can represent, for example, the electroporation catheter 112 (shown in FIG. 1). In the illustrated embodiment, the catheter 150 includes a handle 152, a connector 154 extending proximal from the handle 152, and a tubular shaft 156 extending distal from the handle 152. The shaft 156 includes a non-rigid, naturally straight proximal portion 158 and a deflectable distal portion 160, wherein the distal portion 160 is configured for placement and manipulation within a in a target area of a heart of the patient 102 (shown in FIG. 1). The distal portion 160 includes a deflection region 162 and an end region 164. The end region 164 is another straight section includes an electrode 166 that is located the distal end of the catheter 112. The connector 154 is selectively connectable to other equipment (e.g., the electroporation console 116, shown in FIG. 1) that can send or receive electrical signals to or from the electrode 116.

In the illustrated embodiment, the handle 152 includes an actuator 168 that is configured for steering the catheter 112. The physician can manipulate the actuator 168 to control the amount and direction of deflection of the deflection region 162. Such deflection can be analyzed using a Cartesian coordinate system. In an undeflected state (i.e., a straight configuration), the shaft 156 (i.e., the proximal portion 158 and the distal portion 160) extends along a Y-axis, as shown in FIG. 2A. Perpendicular to the Y-axis are the X-axis and the Z-axis, and in some embodiments, the deflection region 162 is deflectable in the YZ-plane but not in the XY-plane.

FIG. 2B shows the catheter 150 in a first deflected configuration. In the illustrated embodiment, the physician has manipulated the actuator 168 to deflect the deflection region 162 in a counterclockwise direction A so that the end region 164 is positively offset from the Y-axis and is generally oriented in the -Y direction. As will be explained below, the deflection of the catheter 150 occurs substantially solely in the YZ-plane and not substantially in the XY-plane. However, such uniplanar curvature can be affected by the vasculature of the patient 102 since the vasculature can impart forces to the sides of the catheter 150 that can move the bend out of plane.

FIG. 2C shows the catheter 150 in a second deflected configuration. In the illustrated embodiment, the physician has manipulated the actuator 168 to deflect the deflection region 162 in a clockwise direction B so that the end region 164 is negatively offset from the Y-axis and is generally oriented in the -Y direction. As will be explained below, the deflection of the catheter 150 occurs substantially solely in the YZ-plane and not substantially in the XY-plane. However, such uniplanar curvature can be affected by the vasculature of the patient 102 since the vasculature can impart forces to the sides of the catheter 150 that can move the bend out of plane.

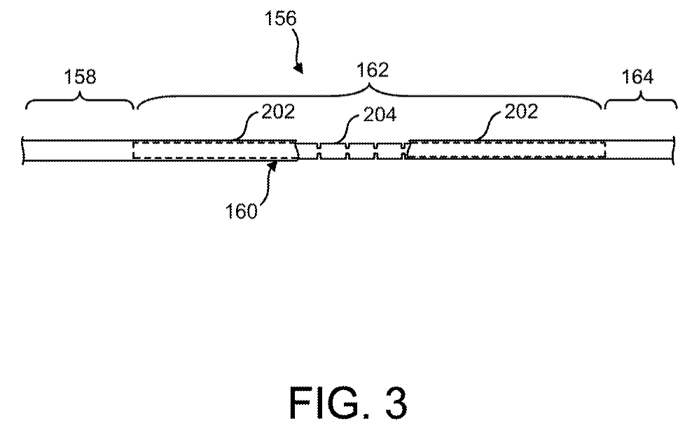

FIG. 3 is a side view of the shaft 156. In the illustrated embodiment, the shaft 156 includes an outer tubular jacket 202, which has been partially cut away to show an articulation member 204 inside of the jacket 202. In some embodiments, the jacket 402 is comprised of a braided polymer material reinforced with metal or polymeric wires that are woven, knitted, entwined, or otherwise interlaced together. In other embodiments, the jacket 202 is constructed differently, for example, using reinforcing coils to enhance the structural and torsional strength of the jacket 202.

In the illustrated embodiment, the articulation member 204 is positioned in the deflection region 162 which begins at the distal end of the proximal end portion 158 and extends to the proximal end of the end region 164. As will be explained in greater detail below, the articulation member 204 is configured to exhibit a relatively high degree of flexibility in the YZ-plane (shown in FIG. 2C), while at the same time being relatively inflexible in the XY-plane (shown in FIG. 2C). As such, the articulation member 204 facilitates predictable, highly planar deflection of the deflection region 162 by resisting torsional forces on the shaft 156 that would otherwise tend to cause the deflection region 162 to deflect or bend in the XY-plane (or some other plane oriented transversely to the YZ-plane).

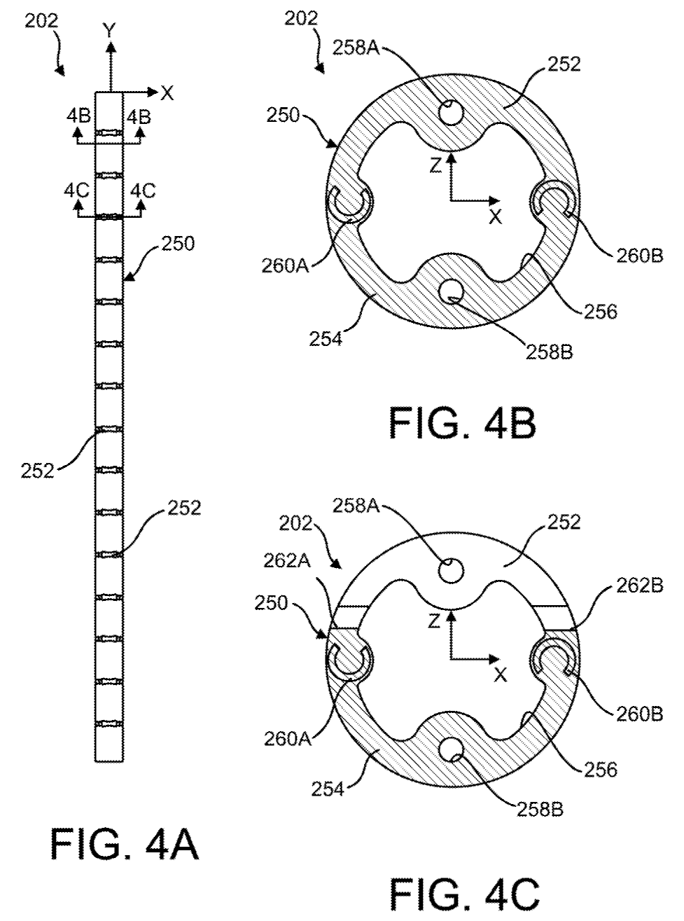

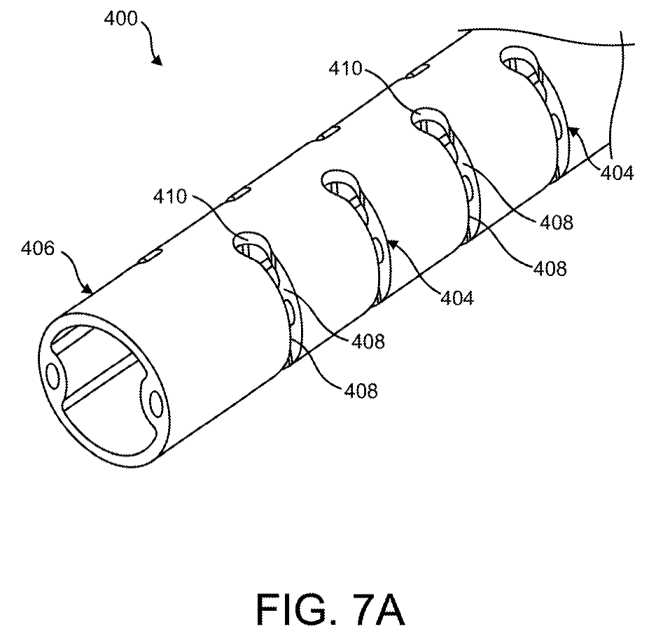

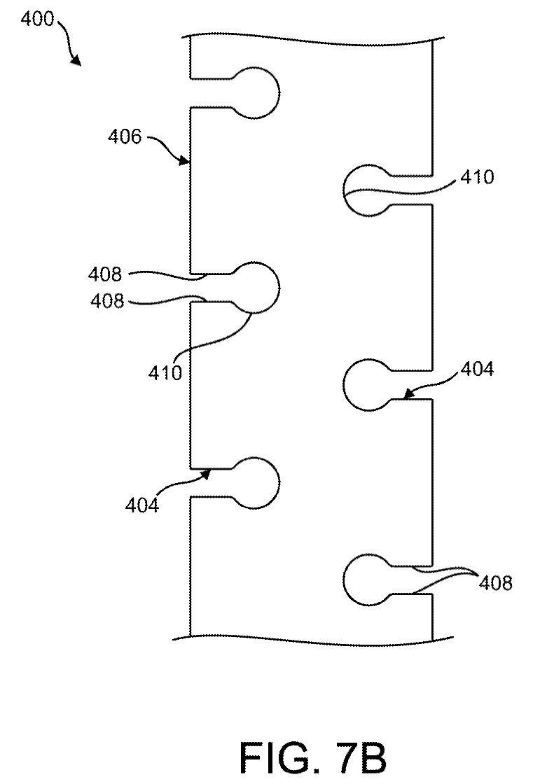

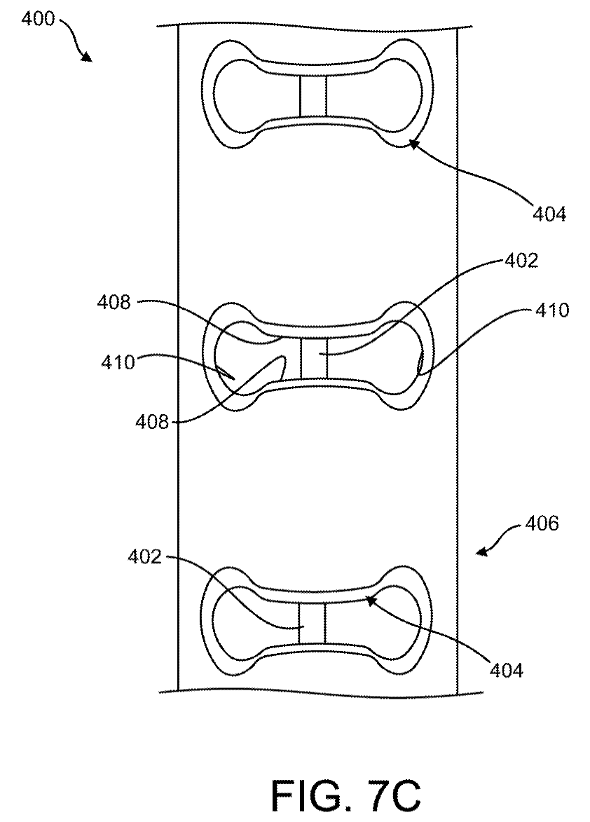

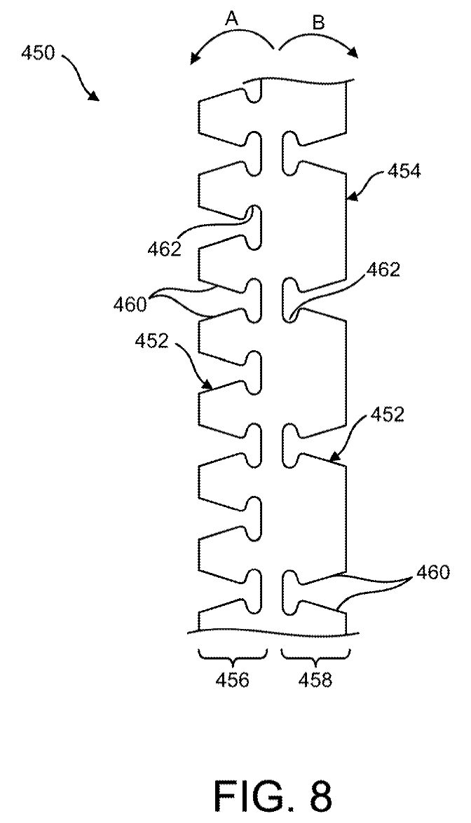

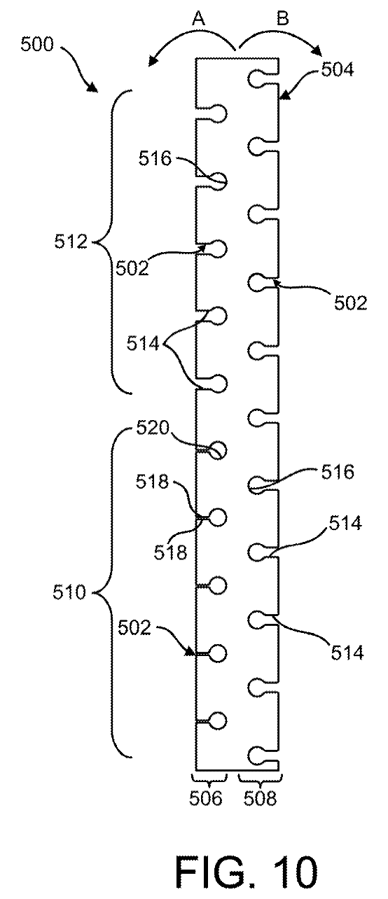

FIG. 4A shows a top view of the articulation member 204. In the illustrated embodiment, the articulation member 204 is a longitudinally extending tube 250 with an array of openings 252 disposed along the tube 250. In some embodiments, the openings 252 are evenly longitudinally spaced apart from each other along the entire length of the tube 250. In other embodiments, the openings 252 extend along only part of the length of the tube 250 and/or are unevenly longitudinally spaced apart from each other. In some embodiments, all of the openings 252 are the same size and shape. In some embodiments, the openings 252 are V-shaped, U-shaped, bulb-shaped (as shown in FIG. 7A), inverted-T-shaped (as shown in FIG. 8), and/or lollipop-shaped (as shown in FIG. 10).

FIG. 4B shows a cross-sectional view of the articulation member 204. This cross-sectional view does not intersect any of the openings 252, so a wall 254 of the tube 250 forms a closed shape at this location. In the illustrated embodiment, the tube 250 comprises a compliant material, such as, for example, a polymer material (e.g., polyether ether ketone (PEEK), polyurethane (PU) (e.g., Pellethane®), polyimide (PI) (e.g., Aurum^{TM}), polypropylene (PP), or polycarbonate (PC)). The tube 250 includes a central lumen 256 arranged centrally with respect to the wall 254. The central lumen 256 is configured to allow the passage of other implements through the articulation member 204, such as, for example, wires for ablation electrodes, navigational components, temperature sensors (e.g., thermocouples), force sensors, radio-frequency circuitry and/or wires, and/or cooling lumens. The tube 250 also includes a pair of steering wire lumens 258 (i.e., lumens 258A, 258B) that are diametrically opposed to one another. The lumens 258 are positioned in the YZ-plane (shown in FIG. 2C), which is the plane in which the articulation member 204 primarily curves. Each of the lumens 258 are configured to accommodate a steering wire (shown in FIG. 7C) that are connected to the actuator 168 (shown in FIG. 2C).

In the illustrated embodiment, the tube 250 includes a pair of reinforcing members 260 (i.e., members 260A and 260B) that are diametrically opposed to one another. The members 260 are positioned in the XY-plane, which is perpendicular to the plane in which the articulation member 204 primarily curves. The members 260 are embedded in the wall 254 and extend longitudinally along the entire length of the tube 250. The members 260 comprise a stronger material than the rest of the tube 250, such as, for example, coiled metal (e.g., nitinol, stainless steel, or tungsten), a rod or beam of uniform cross section, and/or a metal ribbon. In other embodiments, the tube 250 does not include the reinforcing members 260. In some such embodiments, the central lumen 256 is correspondingly larger (e.g., the central lumen 256 has an hourglass shape instead of a cloverleaf shape), and in other such embodiments, the wall 254 maintains its thickness at the corresponding locations where the members 260 are in the illustrated embodiment.

FIG. 4C shows a cross-sectional view of the articulation member 204. This cross-sectional view intersects one of the openings 252, so the wall 254 of the tube 250 forms an open shape at this location. Lateral edges 262 (i.e., edges 262A and 262B) of the opening 252 are oriented horizontally because the edges 262 are positioned in a plane that is offset from the XY-plane (shown in FIG. 2C).

In some embodiments, the articulation member 204 is manufactured starting with an extruded length of material that includes the lumens 256, 258 but not the openings 252. In one exemplary embodiment, the openings 252 are formed by a subtractive manufacturing process, e.g., laser cutting or mechanical cutting across the material, which is why the lateral edges 262 are straight across the material instead of being, for example, radially oriented. Once all of the openings 252 are formed in the material, the tube 250 is complete.

In other embodiments, other manufacturing techniques may be employed to manufacture the various articulation members. Exemplary such techniques include, without limitation, molding (e.g., injection molding), additive manufacturing processes, and the like. In short, the various articulation member embodiments of the present disclosure are not limited by the manufacturing process used to form them.

As will be explained in further detail herein, in various embodiments, the openings 252 may be configured with geometries that minimize stress concentrations in the wall of the tube 250 during articulation of the articulation member 202. Additionally, as further discussed below, in embodiments, the tube 250 is configured such that the functionality of the reinforcing members 260A, 260B is taken up in the tube 250 itself, and thus the reinforcing members 260A, 260B may be omitted (and consequently, the wall thickness of the tube 250 may be substantially uniform in the corresponding regions thereof.

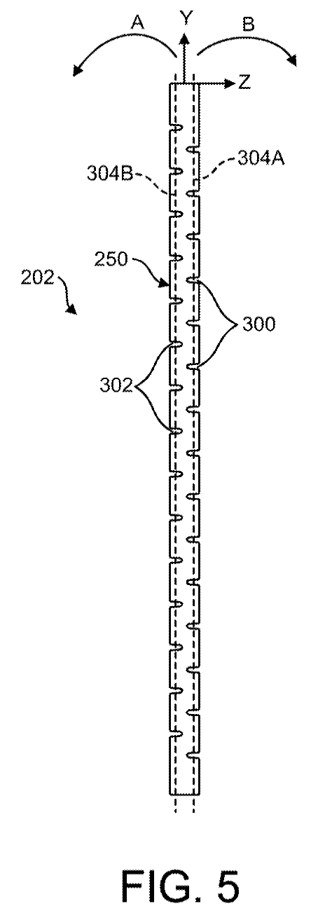

FIG. 5 shows a side view of the articulation member 202. In the illustrated embodiment, there are two sets of openings 252 in the tube 250 that are diametrically opposed to and longitudinally offset from one another. The arrangement of the openings 252 give the tube 250 a serpentine shape when viewed from the side. Specifically, there is an array of top openings 300 on the positive Z-axis side of the tube 250, and there is an array of bottom openings 302 on the negative Z-axis side of the tube 250. The array of top openings 300 is longitudinally offset from the array of bottom openings 302. In some embodiments, there are the same number of top openings 300 and bottom openings 302, although in other embodiments, there is one more of one of the openings 300, 302 than the other of the openings 300, 302. In some embodiments, all of the openings 300, 302 are the same size. In some embodiments, the openings 300 are equally spaced apart from each other, and the openings 302 are equally spaced apart from each other. In some embodiments, the top openings 300 are positioned halfway between the bottom openings 302, and the bottom openings 302 are positioned halfway between the top openings 300.

In the illustrated embodiment, the axes 304 (i.e., axes 304A and 304B) of the steering wire lumens 258 (shown in FIG. 4C) extend longitudinally through the tube 250 in the YZ-plane and are parallel to and equally offset from the Y-axis in opposite directions. The steering wire lumens 258 are configured to receive steering wires (one of which is shown in FIG. 7C) that facilitate the deflection of at least the deflection region 162 (shown in FIG. 3) in a conventional manner. For example, when the steering wire that extends along the axis 304B is pulled by the actuator 168 (shown in FIG. 2C), that steering wire pulls on one side of the tube 250. The tension causes the top openings 300 to expand and the bottom openings 302 to contract, and the articulation member 202 bends in the clockwise direction B. For another example, when the steering wire that extends along the axis 304A is pulled by the actuator 168, that steering wire pulls on one side of the tube 250. The tension causes the bottom openings 302 to expand and the top openings 300 to contract, and the articulation member 202 bends in the counterclockwise direction A.

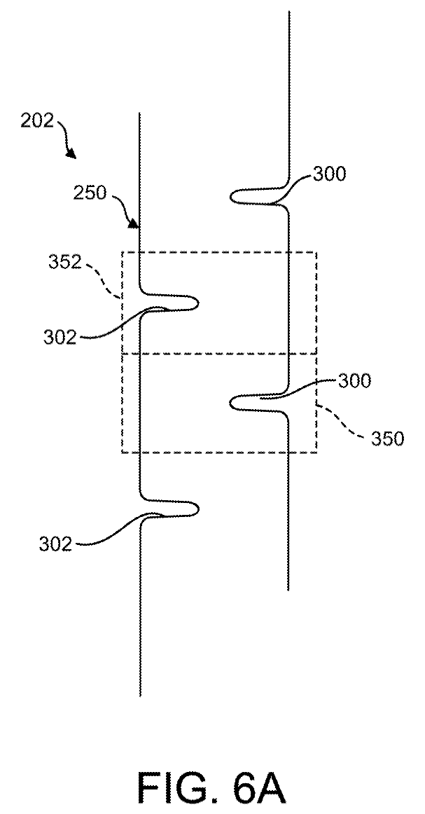

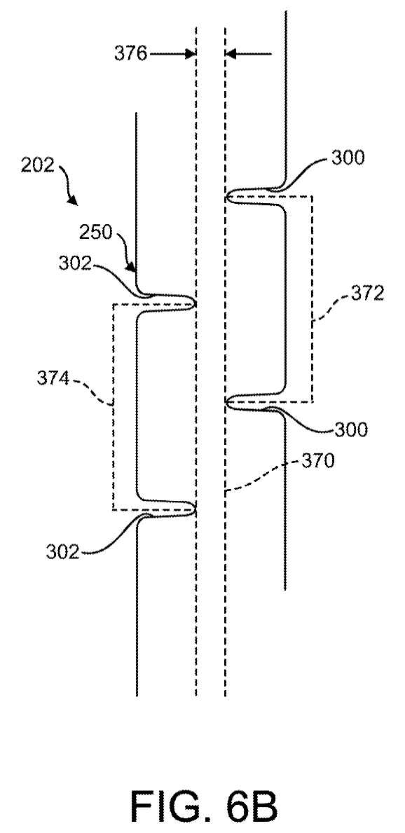

FIG. 6A shows a close-up side view the articulation member 202. As discussed earlier with respect to FIG. 5, the articulation member 202 can be thought of as the tube 250 with openings 300, 302 disposed along all or a portion of its length. However, a different way of understanding the articulation member 202 is as a longitudinally extending array of living hinges 350, 352 with alternating orientations along the length of the tube 250. The living hinges 350 are oriented in one direction (e.g., to the right in FIG. 6A), and the living hinges 352 are oriented in the opposite direction (e.g., to the left in FIG. 6A). In the illustrated embodiment, each living hinge 350 includes a portion of the tube 250 with one of the openings 300, and each living hinge 352 includes a portion of the tube 250 with one of the openings 302. Thus, each living hinge 350, 352 has an opposite orientation from the adjacent living hinge(s) 350, 352.

In the illustrated embodiment, when the articulation member 202 is actuated, each of the living hinges 350 will become more opened or more closed, and each of the living hinges 352 will do the opposite from the living hinges 350. The opening and closing of the living hinges 350, 352, respectively, allow the central lumen 256 (shown in FIG. 4C) to remain open as the articulation member 200 deflects.

The opposing orientations (i.e., 180° apart) of the living hinges 350, 352 means that the deflection of the articulation member 202 occurs in only the deflection plane (e.g., the YZ-plane, shown in FIG. 2C). At the same time, the articulation member 202 is relatively inflexible in the orthogonal plane (e.g., the XY-plane). As such, the living hinges 350, 352 facilitate the articulation member 202 to have predictable, highly planar deflection of the deflection region 162 (shown in FIG. 3) by resisting torsional forces on the articulation member 202 that would otherwise tend to cause the deflection region 162 to deflect or bend in, for example, the XY-plane (or some other plane oriented transversely to, for example, the YZ-plane). In some embodiments, the living hinges 350, 352 are configured to maintain the deflection of the articulation member 202 within ±10° of the deflection plane under normal conditions in the clinical setting (shown in FIG. 1).

FIG. 6B shows a close-up side view the articulation member 202. As discussed earlier with respect to FIG. 5, the articulation member 202 can be thought of as the tube 250 with openings 300, 302 disposed along all or a portion of its length. However, yet another different way of understanding the articulation member 202 is as two longitudinally extending beams 370 that are diametrically opposed to each other (so only one beam 370 is visible in FIG. 6B), and the beams 370 are joined on alternating sides by cylindrical shell sectors 372, 374. In the illustrated embodiment, the beams 370 are longitudinally straight, continuous sections that extend between the innermost extents of the openings 300, 302, which is possible because the openings 300, 302 do not extend past the center of the tube 250. In other words, the openings 300, 302 do not circumferentially overlap each other. In some embodiments, the width 376 of the beams 370 are the same and are between about 0.35 mm (0.014 in.) and about 2.0 mm (0.080 in.), between about 0.51 mm (0.020 in.) and about 2.0 mm (0.080 in.), or between about 0.35 mm (0.014 in.) and about 1.5 mm (0.059 in.). In some such embodiments, the diameter of the shaft 156 (shown in FIG. 3) is about 2.834 mm (8.5 Fr).

In the illustrated embodiment, the cylindrical shell sectors 372, 374 have slightly less than a half-pipe shape. The beams 370 and the cylindrical shell sectors 372 define the openings 300, and the beams 370 and the cylindrical shell sectors 374 define the openings 302. Thus, the sectors 372 are separated from each other by the openings 300, and the sectors 374 are separated from each other by the openings 302. The cylindrical shell sectors 372 are diametrically opposed to and longitudinally offset from the cylindrical shell sectors 374. Thus, each sector 372 longitudinally overlaps two adjacent sectors 374, and each sector 374 longitudinally overlaps two adjacent sectors 372 (although at the last sectors 372, 374 at the ends of the articulation member 202 may longitudinally overlap only one of the opposite sectors 372, 374, respectively).

When the articulation member 202 deflects, the beams 370 bend. In the illustrated embodiment, if the deflection is to the right, then the bending of the beams 370 causes the adjacent sectors 372 to move closer together (or perhaps to contact each other) and the adjacent sectors 374 to move farther apart. In such a scenario, the sides of the beams 370 that are closest to the sectors 372 are under a compressive load and the sides of the beams 370 that are closest to the sectors 374 are under a tensile load. In the illustrated embodiment, if the deflection is to the left, then the bending of the beams 370 causes the adjacent sectors 374 to move closer together (or perhaps to contact each other) and the adjacent sectors 372 to move farther apart. In such a scenario, the sides of the beams 370 that are closest to the sectors 374 are under a compressive load and the sides of the beams 370 that are closest to the sectors 372 are under a tensile load. Such compressive and tensile loads cause deformation of the beams 370 as the articulation member 202 deflects.

In the various embodiments, the longitudinal staggering of the openings 300, 302, combined with configuring the openings 300, 302 such that they do not overlap circumferentially, provides an articulating member that exhibits increased resistance to undesired plastic deformation at the hinge portions as compared to existing articulation member configurations in which the openings are longitudinally aligned and/or circumferentially overlap one another.

FIGS. 7A-7C shows an alternative articulation member 400. More specifically, FIG. 7A shows a perspective view of the alternative articulation member 400, FIG. 7B shows a side view of the alternative articulation member 400, and FIG. 7C shows a top view of the alternative articulation member 400. FIGS. 7A-7C will now be discussed in conjunction with one another. As indicated previously, FIG. 7C includes one of the steering elements 402 (e.g., wires, tendons, ribbons made of stainless steel, titanium, MP35N, a suitable alloy, and/or other suitable materials).

In the illustrated embodiment, the articulation member 400 includes a plurality of diametrically opposed and longitudinally staggered openings 404 defined by a tube 406. This arrangement of the openings 404 is the same as or similar to that of the openings 252 (shown in FIG. 4A). However, the openings 404 themselves are different from the openings 252. In particular, the openings 404 are bulb-shaped in that there are two parallel sides 408 that extend orthogonally towards the center of the tube 406, and the openings 404 terminate with a circular cutout 410 that has a larger diameter than the distance between the two parallel sides 408. Thus, the circular cutouts 410 provide stress relief for the tube 406. Such a stress relieving shape prevents plastic deformation of the tube 406 during deflection of the articulation member 400.

As can be seen in FIG. 7A, the articulation member 400 does not include the reinforcing members 260A, 260B (see FIGS. 4B and 4C). Accordingly, in this and other embodiments, the wall thickness of the tube of the articulation member may be generally uniform in the corresponding regions thereof.

FIG. 8 is a side view an alternative articulation member 450. In the illustrated embodiment, the articulation member 450 includes a plurality of openings 452 that are defined by a tube 454. The openings 452 are positioned in two diametrically opposed arrays 456, 458. The array 456 has twice as many openings 452 as the array 458, so every other opening 452 in the array 456 is at the same longitudinal position as one of the openings 452 in the array 458. However, the other openings 452 in the array 456 are longitudinally offset from the openings 452 in the array 458. In other embodiments, the array 456 has more openings 452 than the array 458, although the amount is different than double (i.e., the amount is different than 100% more openings 452). In some such embodiments, there are at least about 20% more, at least about 50% more, at least about 100% more, at least about 150% more, or at least about 200% more. In such embodiments, how frequently (if at all) the openings 452 in the array 456 will longitudinally coincide with the openings 452 in the array 458 will vary based on the geometry of the particular embodiment.

In the illustrated embodiment, each of the openings 452 has an inverted-T-shape in that there are two sides 460 that extend towards each other as they extend towards the center of the tube 454, and the openings 452 terminate with an obround cutout 462 that extends parallel to the longitudinal axis of the tube 454 and has a larger width than the distance between the two sides 460 and their centermost ends. Thus, the obround cutouts 410 provide stress relief for the tube 454. Such a stress relieving shape prevents plastic deformation of the tube 454 during deflection of the articulation member 450.

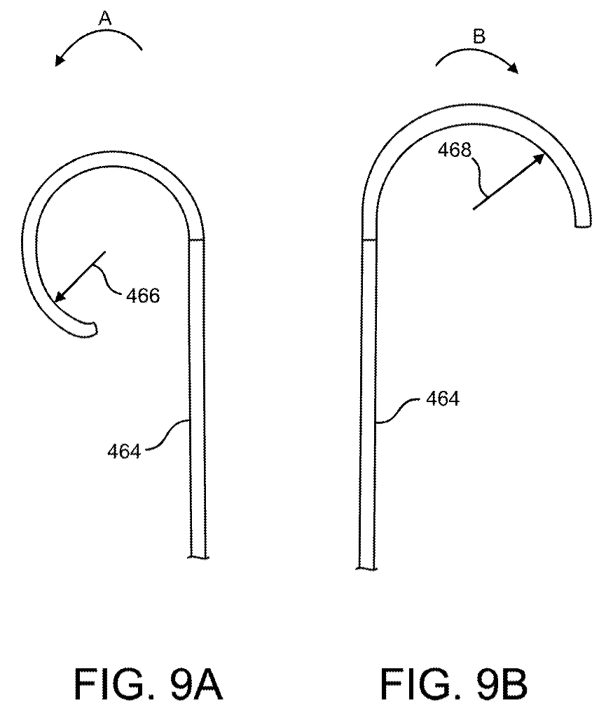

FIG. 9A is a side-view of a portion of an alternative catheter 464 that includes the articulation member 450 (shown in FIG. 8) and is deflected in direction A towards the array 456 of openings 452 (both shown in FIG. 8). FIG. 9B is a side-view of a portion of the alternative catheter 464 that includes the articulation member 450 (shown in FIG. 8) and is deflected in direction B towards the array 458 of openings 452 (both shown in FIG. 8). FIGS. 9A and 9B will now be discussed in conjunction with one another.

In the illustrated embodiment, the increased number of openings 452 in the array 456 allows the catheter 464 to deflect toward the array 456 (as shown in FIG. 9A) with a radius of curvature 466 that is tighter than a radius of curvature 468 when the catheter 464 deflects toward the array 458 (as shown in FIG. 9B). In some embodiments, the radius of curvature 466 is about 80% of the radius of curvature 468. In some embodiments, the radius of curvature 466 is no more than about 90% of the radius of curvature 468, no more than about 75% of the radius of curvature 468, no more than about 60% of the radius of curvature 468, no more than about 50% of the radius of curvature 468, no more than about 40% of the radius of curvature 468, or no more than about 30% of the radius of curvature 468.

FIG. 10 is a side view a portion of an alternative articulation member 500. In the illustrated embodiment, the articulation member 500 includes a plurality of openings 502 that are defined by a tube 504. The openings 502 are positioned in two diametrically opposed and longitudinally staggered arrays 506, 508. Furthermore, the array 506 is divided into two longitudinally adjacent sets 510, 512 with the set 510 being on the proximal portion of the articulation member 500 and the set 512 being on the distal portion of the articulation member 500. The openings 502 in the array 508 and the set 512 are the same size and shape, wherein the openings 502 are bulb-shaped in that there are two parallel sides 514 that extend orthogonally towards the center of the tube 504, and the openings 502 terminate with a circular cutout 516 that has a larger diameter than the distance between the two parallel sides 514.

In the illustrated embodiment, the openings 502 in the set 510 are different from the openings 502 in the array 508 and the set 512. The openings 502 in the set 510 are lollipop-shaped in that there are two parallel sides 518 that extend orthogonally towards the center of the tube 504, and the openings 502 terminate with a circular cutout 520. However, there is a closed, substantially zero, or very small gap between the pairs of sides 518 because the pairs of sides 518 are in contact with or are spaced very closely to each other when the articulation member 500 is in an undeflected state. In some embodiments, the spaces between the pairs of sides 514 are at least about five times, at least about ten times, at least about twenty times, or at least about fifty times larger than the spaces between the pairs of sides 518. In some embodiments, the length of the proximal portion of the articulation member 500 that includes the set 512 is between about 10 mm and about 20 mm or is about 15 mm long.

In the illustrated embodiment, the proximal portion of the articulation member 500 that includes the set 510 is substantially inflexible in the direction towards the set 510 (i.e., in direction A). In contrast, the distal portion of the articulation member 500 that includes the set 512 is substantially flexible in the direction towards the set 512 (i.e., in direction A). Furthermore, the entire length of the articulation member 500 is substantially flexible in the direction towards the array 508. Thus, the effective length of the articulation member 500 is essentially shorter when deflecting in the direction A compared to the effective length of the articulation member 500 when deflecting in the direction B, which results in an asymmetric bending profile for the articulation member 500. In other embodiments, the openings 502 in the set 510 are selectively located along the articulation member 500 so as to allow the articulation member 500 to assume other asymmetric bending profiles.

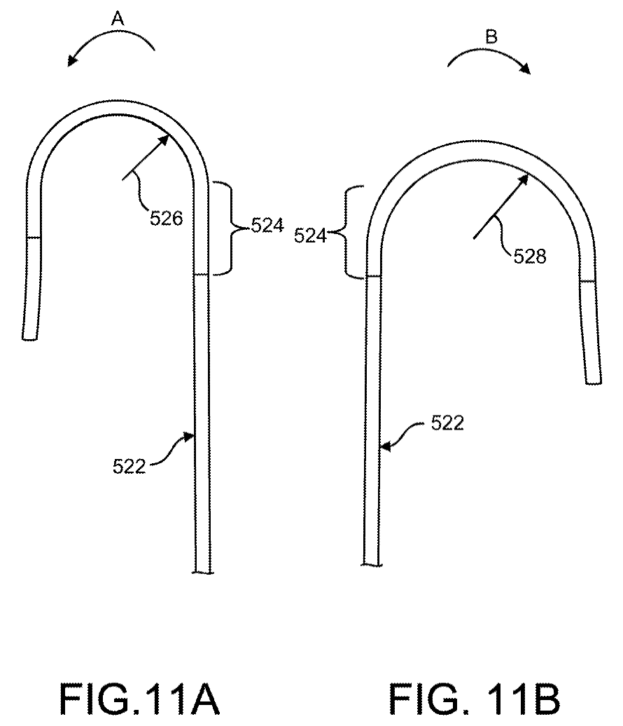

FIG. 11A is a side-view of a portion of an alternative catheter 522 that includes the articulation member 500 (shown in FIG. 10) and is deflected in the direction A towards the array 506 of openings 502 (both shown in FIG. 10). FIG. 11B is a side-view of a portion of the alternative catheter 522 that includes the articulation member 500 (shown in FIG. 10) and is deflected in the direction B towards the array 508 of openings 502 (both shown in FIG. 10). FIGS. 11A and 11B will now be discussed in conjunction with one another.

In the illustrated embodiment, a longitudinal portion 524 of the catheter 522 includes the set 510 (shown in FIG. 10) of openings 502. As shown in FIG. 11A, when a deflection force is applied by steering element 402 (shown in FIG. 7C) that extends through the array 506 of openings 502, the openings 502 in the array 506 close. While the openings 502 in the set 512 have substantial gaps between their sides 514 (shown in FIG. 10), the openings 502 there are little to no gaps between their sides 518 (shown in FIG. 10), so there is little to no deflection in the longitudinal portion 524 when the (leftmost) steering element 402 is actuated. The catheter 522 deflects in the direction A to a first curved shape, wherein the catheter 522 begins to substantially curve at the distal end of the longitudinal portion 524.

As shown in FIG. 11B, when a deflection force is applied by steering element 402 (shown in FIG. 7C) that extends through the array 508 of openings 502, the openings 502 in the array 508 close. The openings 502 in the array 508 all have substantial gaps between their sides 514 (shown in FIG. 10), so there is a standard amount of deflection in the longitudinal portion 524 when the (rightmost) steering element 402 is actuated. The catheter 522 deflects in the direction B to a second curved shape, wherein the catheter 522 begins to substantially curve at the proximal end of the longitudinal portion 524.

The result is that a radius of curvature 526 of the first curved shape is smaller than a radius of curvature 528. In some embodiments, the radius of curvature 526 is between about 4 mm and about 28 mm, and in some embodiments, the radius of curvature 528 is between about 4 mm to about 40 mm. Thereby, a single catheter 522 is configured to have different steering curvatures depending on the which direction the physician orients the catheter 522. In some embodiments, the radius of curvature 526 is about thirty percent tighter than the radius of curvature 528. The difference in radii of curvature 526, 528 aids the physician in navigating the various patient anatomy and can prevent the need to use multiple different catheters for a single procedure.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A medical device comprising:
a handle; and
a tubular shaft having a proximal portion extending from the handle, and a distal portion having a distal end and a deflection region, the shaft defining a longitudinal axis and including:
an outer tubular jacket; and
an articulation member disposed within the jacket in the deflection region, the articulation member having a tube that defines a plurality of openings, wherein:
the openings are arranged in a first array and a second array;
the openings in the first array are diametrically opposed to the openings in the second array;
the openings in the first array do not circumferentially overlap the openings in the second array; and
the first array is longitudinally offset from the second array.

2. The medical device of claim 1, wherein the openings have a stress relieving shape.

3. The medical device of claim 2, wherein the openings are bulb-shaped, inverted-T-shaped, and/or lollipop-shaped.

4. The medical device of claim 1, wherein:
each of the openings comprises a set of sides;
each of a first set of the openings has a first gap between the set of sides; and
each of a second set of the openings has a second gap between the set of sides;
wherein the first gap is smaller than the second gap.

5. The medical device of claim 4, wherein the first gap is substantially zero.

6. The medical device of claim 4, wherein first array of openings comprises the first set of the openings and the second set of openings.

7. The medical device of claim 1, wherein the first array of openings comprises at least 20% more openings than the second array of openings.

8. The medical device of claim 1, further comprising:
first and second steering wire lumens extending through the tubular shaft to a location distally beyond the articulation member; and
first and second steering wires received, respectively, within the first and second steering wire lumens, the first and second steering wires each connected to a steering actuator disposed within the handle and to the distal portion of the shaft at a location distal of the articulation region, wherein the first steering wire is configured to apply a first deflection force to the distal portion of the shaft to cause the deflection region to articulate in the first direction, and wherein the second steering wire is configured to apply a second deflection force to the distal portion of the shaft to cause the deflection region to articulate in the second direction.

9. A medical device comprising:
a handle; and
a tubular shaft having a proximal portion extending from the handle, and a distal portion having a distal end and a deflection region, the shaft defining a longitudinal axis and including:
an outer tubular jacket; and
an articulation member disposed within the jacket in the deflection region, the articulation member having a tube that defines a plurality of openings, wherein:
the tube comprises a first straight beam, a second straight beam, a first plurality of cylindrical shell sectors that join the first straight beam and the second straight beam, and a second plurality of cylindrical shell sectors that join the first straight beam and the second straight beam;
the first plurality of cylindrical shell sectors is diametrically opposed to and longitudinally offset from the second plurality of cylindrical shell sectors.

10. The medical device of claim 9, wherein the openings have a stress relieving shape.

11. The medical device of claim 10, wherein the openings are bulb-shaped, inverted-T-shaped, and/or lollipop-shaped.

12. The medical device of claim 9, wherein:
each of the openings comprises a set of sides;
each of a first set of the openings has a first gap between the set of sides; and
each of a second set of the openings has a second gap between the set of sides;
wherein the first gap is smaller than the second gap.

13. The medical device of claim 12, wherein the first gap is substantially zero.

14. The medical device of claim 12, wherein the first set of the openings and the second set of openings are defined by first plurality of cylindrical shell sectors.

15. The medical device of claim 9, wherein the first plurality of cylindrical shell sectors comprises at least 20% more cylindrical shell sectors than the second plurality of cylindrical shell sectors.

16. The medical device of claim 9, further comprising:
first and second steering wire lumens extending through the tubular shaft to a location distally beyond the articulation member; and
first and second steering wires received, respectively, within the first and second steering wire lumens, the first and second steering wires each connected to a steering actuator disposed within the handle and to the distal portion of the shaft at a location distal of the articulation region, wherein the first steering wire is configured to apply a first deflection force to the distal portion of the shaft to cause the deflection region to articulate in the first direction, and wherein the second steering wire is configured to apply a second deflection force to the distal portion of the shaft to cause the deflection region to articulate in the second direction.

17. A medical device comprising:
a handle; and
a tubular shaft having a proximal portion extending from the handle, and a distal portion having a distal end and a deflection region, the shaft defining a longitudinal axis and including:
an outer tubular jacket; and
an articulation member disposed within the jacket in the deflection region, the articulation member having a tube that defines a plurality of openings and defines a longitudinally extending array of living hinges, wherein:
each living hinge includes one of the plurality of openings;
each living hinge has a opposite orientation from an adjacent living hinge;
the plurality of openings are arranged in a first array and a second array; and
the openings in the first array do not circumferentially overlap the openings in the second array.

18. The medical device of claim 17, wherein the openings are bulb-shaped, inverted-T-shaped, and/or lollipop-shaped.

19. The medical device of claim 17, wherein:
each of the openings comprises a set of sides;
each of a first set of the openings has a first gap between the set of sides; and
each of a second set of the openings has a second gap between the set of sides;
wherein the first gap is smaller than the second gap.

20. The medical device of claim 17, wherein the first array of openings comprises at least 20% more openings than the second array of openings.
